(19) United States
(12) Patent Application Publication
(10) Pub. No.: US 2017/0029869 A1
(43) Pub. Date: Feb. 2, 2017

(54) LYOPHILIZED MATERIAL PRODUCTION METHOD

(71) Applicant: Seiko Epson Corporation, Tokyo (JP)

(72) Inventor: Kotaro IDEGAMI, Chino (JP)

(21) Appl. No.: 15/218,185

(22) Filed: Jul. 25, 2016

(30) Foreign Application Priority Data
Jul. 29, 2015 (JP) 2015-149350

(51) Int. Cl. C12Q 1/68

(52) U.S. Cl. CPC C12Q 1/6806; C12Q 1/686

(57) ABSTRACT

A lyophilized material production method according to the invention is a lyophilized material production method for producing a lyophilized material by lyophilizing a reagent containing an additive at a lyophilization temperature, and includes a freezing step in which the reagent containing an additive is cooled with a low-temperature liquid whose temperature is lower than the lyophilization temperature, a maintaining step in which the temperature of the frozen reagent is increased to a temperature higher than the boiling point of the low-temperature liquid and the reagent is maintained at the temperature for a predetermined time, and a lyophilization step in which the reagent having been maintained for a predetermined time after increasing the temperature of the reagent is dried at the lyophilization temperature under reduced pressure.

### BACKGROUND

1. Technical Field

The present invention relates to a lyophilized material production method.

2. Related Art

In the field of biochemistry, a PCR (Polymerase Chain Reaction) technique has been established. Recently, amplification accuracy and detection sensitivity of a PCR method have been improved, and it has become possible to amplify an extremely small amount of a sample (DNA or the like) and to perform detection and analysis of the sample. The PCR is a method for amplifying a target nucleic acid by subjecting a solution (reaction solution) containing a nucleic acid (target nucleic acid) to be amplified and a reagent to thermal cycling. For the thermal cycling in PCR, a method for performing thermal cycling at temperatures in two stages or three stages is generally used.

It is generally very difficult to store a nucleic acid amplification reaction reagent to be used in PCR for a long period of time because an enzyme to be used in the reagent shows low storage performance. In particular, a reverse transcriptase cannot be kept for even 1 day in a state of an aqueous solution even if the aqueous solution is stored at 4° C., and the activity is decreased. Therefore, it has been known that the storage stability of the reagent is improved by adding an additive to the reagent and lyophilizing the reagent (see, for example, JP-T-2000-514298 (PTL 1)). Here, by including trehalose at a concentration of 8 to 12% (weight/volume) with respect to the reaction mixture, the storage stability of the lyophilized reaction mixture is improved.

However, it is difficult to redissolve a lyophilized material obtained by the above-mentioned technique, and it is necessary to perform mixing using a vortex mixer, and also the time required for redissolving the material is generally several minutes or more. In addition, if a genetic testing device is going to be provided with a vortex mixer, the mechanism of the device becomes complicated, which is a problem when an automatic testing device is constructed.

### SUMMARY

An advantage of some aspects of the invention is to provide a lyophilized material production method for producing a lyophilized material which has improved resolubility and can be easily redissolved in about a few seconds by solving at least part of the problem described above.

The invention can be implemented as the following aspects or application examples.

### Application Example 1

This application example is directed to a lyophilized material production method for producing a lyophilized material by lyophilizing a reagent containing an additive at a lyophilization temperature, including a freezing step in which the reagent containing an additive is cooled with a low-temperature liquid whose temperature is lower than the lyophilization temperature, a maintaining step in which the temperature of the frozen reagent is increased to a temperature higher than the boiling point of the low-temperature liquid and the reagent is maintained at the temperature for a predetermined time, and a lyophilization step in which the reagent having been maintained for a predetermined time after increasing the temperature of the reagent is dried at the lyophilization temperature under reduced pressure.

According to the lyophilized material production method according to this application example, by including a freezing step in which the reagent containing an additive is cooled with a low-temperature liquid whose temperature is lower than the lyophilization temperature and a maintaining step in which the temperature of the frozen reagent is increased to a temperature higher than the boiling point of the low-temperature liquid and the reagent is maintained at the temperature for a predetermined time before the lyophilization step, the resolubility of the lyophilized material produced is improved, and a lyophilized material which can be easily redissolved in about a few seconds can be produced.

### Application Example 2

In the lyophilized material production method according to the application example, the liquid may be liquid nitrogen.

### Application Example 3

In the lyophilized material production method according to the application example, the additive may contain trehalose.

### Application Example 4

In the lyophilized material production method according to the application example, the reagent may contain a reverse transcriptase.

### Application Example 5

In the lyophilized material production method according to the application example, the reagent may contain at least one member selected from a polymerase, dNTP, a primer, and a probe.

### Application Example 6

In the lyophilized material production method according to the application example, the additive may be contained in an amount of 47 mass % or more and 95 mass % or less with respect to the total mass of the solids contained in the lyophilized material.

### Application Example 7

In the lyophilized material production method according to the application example, the additive may be contained in an amount of 78 mass % or more and 85 mass % or less with respect to the total mass of the solids contained in the lyophilized material.

### Application Example 8

In the lyophilized material production method according to the application example, the method may include an addition step in which a hydrophobic liquid is added after the lyophilization step.

### Application Example 9

In the lyophilized material production method according to the application example, the hydrophobic liquid may be a silicone oil.

### BRIEF DESCRIPTION OF THE DRAWINGS

The invention will be described with reference to the accompanying drawings, wherein like numbers reference like elements.

FIG. 1 is an SEM image of a lyophilized material of Example 1.

FIG. 2 is an SEM image of a lyophilized material of Comparative Example 1.

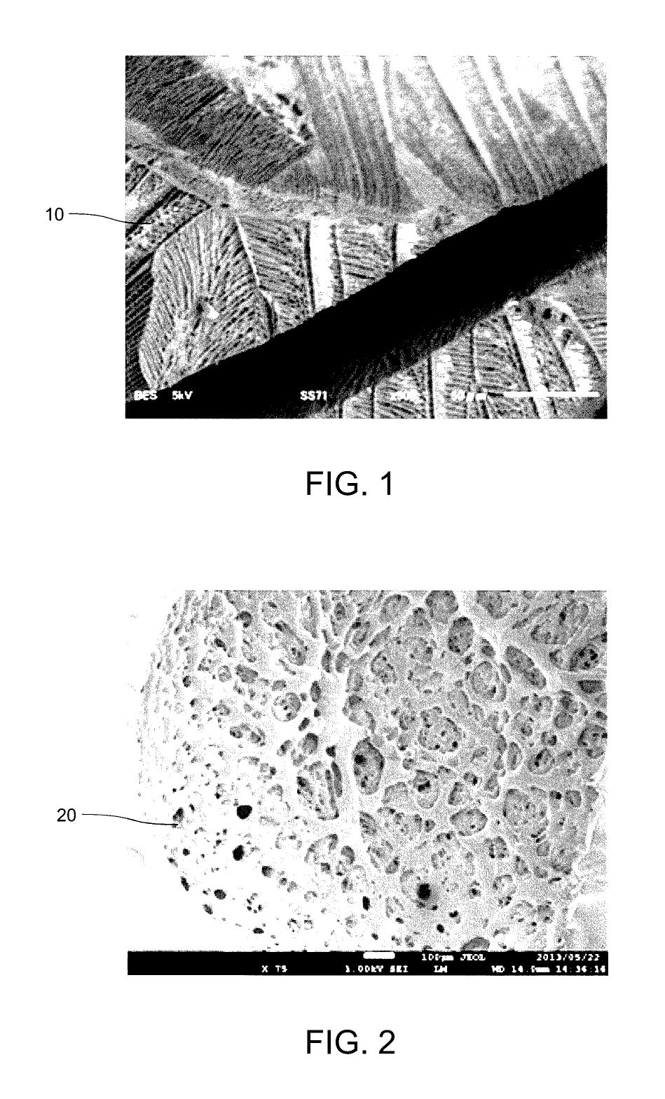

### DESCRIPTION OF EXEMPLARY EMBODIMENTS

Hereinafter, preferred embodiments of the invention will be described. The below-mentioned embodiments describe an example of the invention. Further, the invention is not limited to the following embodiments and also includes various modifications made within the scope not changing the gist of the invention.

### 1. Lyophilized Material Production Method

The lyophilized material production method according to this embodiment is a lyophilized material production method for producing a lyophilized material by lyophilizing a reagent containing an additive at a lyophilization temperature, and includes a freezing step in which the reagent containing an additive is cooled with a low-temperature liquid whose temperature is lower than the lyophilization temperature, a maintaining step in which the temperature of the frozen reagent is increased to a temperature higher than the boiling point of the low-temperature liquid and the reagent is maintained at the temperature for a predetermined time, and a lyophilization step in which the reagent having been maintained for a predetermined time after increasing the temperature of the reagent is dried at the lyophilization temperature under reduced pressure. Hereinafter, the respective steps of the lyophilized material production method according to this embodiment will be described.

1.1. Freezing Step (preliminary freezing step prior to lyophilization step)

The lyophilized material production method according to this embodiment includes a freezing step in which the reagent containing an additive is cooled with a low-temperature liquid whose temperature is lower than the lyophilization temperature. This freezing step is performed for preliminarily freezing a reagent to be lyophilized using a low-temperature liquid whose temperature is lower than the lyophilization temperature prior to the below-mentioned lyophilization step (hereinafter also referred to as “preliminary freezing”), and by including this step, the average opening diameter of micropores of the lyophilized material produced is made small. This preliminary freezing step is performed by, for example, placing a buffer for a nucleic acid amplification reaction and a reagent in an amount for one reaction in a container to be used for a container for a nucleic acid amplification reaction.

### 1.1.1. Reagent

Here, the reagent containing an additive to be preliminarily lyophilized refers to an aqueous solution in which a reagent to be used for a nucleic acid amplification reaction and an additive are dissolved in, for example, sterile water, purified water such as distilled water or ion exchanged water, or such water.

Examples of the reagent to be used for a nucleic acid amplification reaction include a polymerase and dNTP, however, it is preferred to contain a primer for a nucleic acid amplification reaction and/or a probe for a nucleic acid amplification reaction, or a buffer. The reagent may contain a reverse transcriptase. In such a case, a primer for reverse transcription may be contained aside from the primer for a nucleic acid amplification reaction, or may be shared with the primer for a nucleic acid amplification reaction.

The polymerase is not particularly limited, however, examples thereof include a DNA polymerase. As the DNA polymerase, a heat resistant enzyme or an enzyme for PCR is preferred, and there are a lot of commercially available products, for example, Taq polymerase, Tfi polymerase, Tth polymerase, modified forms thereof, and the like, however, a DNA polymerase capable of performing hot start is preferred.

dNTP represents a mixture of four types of deoxyribonucleotide triphosphates. That is, dNTP represents a mixture of dATP (deoxyadenosine triphosphate), dCTP (deoxycytidine triphosphate), dGTP (deoxyguanosine triphosphate), and dTTP (thymidine triphosphate).

Also the reverse transcriptase is not particularly limited, and for example, a reverse transcriptase derived from avian myeloblast virus, Ras-associated virus type 2, mouse Moloney murine leukemia virus, or human immunodefficiency virus type 1, or the like can be used, however, a heat resistant enzyme is preferred. There are also a lot of commercially available products of the heat resistant enzyme, and therefore, such a product can be used.

Here, the concentrations of a polymerase, dNTP, a probe, a primer, a buffer, and a salt in the reagent are appropriately set according to the reaction to be used, however, for example, the concentration of dNTP may be set to 10 to 1000 mM, preferably 100 to 500 mM, the concentration of Mg²⁺ may be set to 1 to 100 mM, preferably 5 to 10 mM, and the concentration of Cl⁻ may be set to 1 to 2000 mM, preferably 200 to 700 mM. The total ion concentration is not particularly limited, but may be higher than 50 mM, preferably higher than 100 mM, more preferably higher than 120 mM, further more preferably higher than 150 mM, still further more preferably higher than 200 mM. The upper limit thereof is preferably 500 mM or less, more preferably 300 mM or less, further more preferably 200 mM or less. The oligonucleotides for primers are used at 0.1 to 20 mM, respectively.

### 1.1.2. Additive

In this embodiment, the additive to be added to the reagent is contained in an amount of 47 mass % or more and 95 mass % or less with respect to the total mass of the solids contained in the lyophilized material produced. The enzyme or the like to be used in the reagent has poor storage stability, and therefore, in order to maintain the activity thereof for a long period of time, the additive is added in a predetermined amount.

The additive is not particularly limited as long as it is a material which has a function of maintaining a nucleic acid amplification reaction without inactivating the lyophilized enzyme or the like when the reagent to be used for a nucleic acid amplification reaction is lyophilized, that is, a material which has excellent storage stability, however, examples thereof include a sugar. The sugar is not particularly limited as long as it is a sugar which functions as a cryoprotective agent, but is preferably a sugar which is easily dissolved in water and does not react with other substances such as an enzyme. Examples of the sugar include sucrose, trehalose, raffinose, and melezitose, which are nonreducing sugars, among disaccharides and trisaccharides. Among these sugars, it is particularly preferred to use trehalose because the function as a cryoprotective agent is high.

In this embodiment, the content of the additive is 47 mass % or more and 95 mass % or less, preferably 78 mass % or more and 85 mass % or less with respect to the total mass (100 mass %) of the solids contained in the lyophilized material. When the content of the additive is 47 mass % or more and 95 mass % or less, the storage stability of the lyophilized material produced is favorable, and the reaction inhibition is less likely to occur when the lyophilized material is redissolved and a nucleic acid amplification reaction is performed.

### 1.1.3. Preliminary Freezing

In this embodiment, in the preliminary freezing procedure in which the reagent containing the additive is cooled with a low-temperature liquid, the low-temperature liquid is not particularly limited as long as it is a liquid whose temperature is lower than the temperature in the below-mentioned lyophilization step and is capable of freezing the reagent, however, for example, liquid nitrogen can be used. Liquid nitrogen has a boiling point at 1 atm of −196° C., and can rapidly decrease the temperature of the reagent containing the additive to be lyophilized and freeze the reagent. Due to this, the lyophilized material obtained by being subjected to preliminary freezing has micropores having a small average opening diameter, and therefore, the resolubility of the lyophilized material is improved, and the lyophilized material can be easily redissolved in about a few seconds.

The temperature in the preliminary freezing step is not particularly limited as long as it is lower than the temperature in the lyophilization step as described above, and also lower than the collapse temperature of the reagent to be frozen, but is preferably from −70° C. to −200° C. Further, the time for the preliminary freezing is not particularly limited as long as it is a time for sufficiently freezing the reagent, however, in the case of using liquid nitrogen, when the amount of the solution is about 2 μL and the solution can be brought into direct contact with liquid nitrogen, preliminary freezing can be sufficiently performed even for about 5 minutes. Incidentally, in order to rapidly cool the reagent, it is preferred to bring the reagent to be frozen into direct contact with the low-temperature liquid such as liquid nitrogen.

As the liquid which can be used other than liquid nitrogen, for example, a liquid such as ethanol or acetone cooled with dry ice can be used. In this case, the temperature of cold ethanol or cold acetone is from −78° C. to −80° C.

### 1.2. Maintaining Step

The lyophilized material production method according to this embodiment includes a maintaining step in which the temperature is increased to a temperature higher than the boiling point of the low-temperature liquid used in the above-mentioned preliminary freezing step and maintained for a predetermined time. The maintaining step is a step intended to sufficiently vaporize the low-temperature liquid so that the low-temperature liquid does not remain in the reagent by increasing the temperature of the low-temperature liquid used in the preliminary freezing step.

The temperature in the maintaining step is not particularly limited as long as it is higher than the boiling point of the low-temperature liquid used in the freezing step, and also lower than the collapse temperature of the reagent to be frozen, but is preferably from −70° C. to −80° C. Further, the time for the maintaining step is not particularly limited as long as it is a time for sufficiently vaporizing the low-temperature liquid contained in the reagent.

In this embodiment, by including the maintaining step prior to the lyophilization step, the low-temperature liquid used in the preliminary freezing step is prevented from hindering lyophilization in the subsequent lyophilization step, and thus, the lyophilized material can be appropriately produced.

### 1.3. Lyophilization Step

The lyophilized material production method according to this embodiment includes a lyophilization step in which the reagent is lyophilized under reduced pressure. The lyophilization step is a step in which the reagent having been subjected to the maintaining step is lyophilized, and after the low-temperature liquid is vaporized in the maintaining step, the reagent is further left under reduced pressure for a predetermined time to vaporize water in the reagent containing the additive, whereby the reagent is lyophilized.

The temperature when performing the lyophilization is not particularly limited, but is preferably set to a temperature lower than the collapse temperature of the reagent to be lyophilized, and is preferably −80° C. or higher. Further, the pressure when reducing the pressure is not particularly limited, but is preferably 100 mmHg or less, and most preferably 20 mmHg or less. Further, the time for the lyophilization step is not particularly limited, but is preferably from 2 to 24 hours, and most preferably about 8 hours.

In this embodiment, by lyophilizing the reagent in the container to be used for the container for a nucleic acid amplification reaction in this manner, the reagent containing the additive is solidified and adhered to the bottom of the container.

### 1.4. Addition Step

It is preferred that the lyophilized material production method according to this embodiment further includes an addition step in which a hydrophobic liquid is added after the lyophilization step. The lyophilized material solidified and adhered to the bottom of the container is weak in water and cannot be stored stably for a long period of time in the presence of water. Therefore, by covering the lyophilized material with a hydrophobic liquid dehydrated beforehand, the lyophilized material is less likely to be exposed to a water molecule, and thus, the storage stability performance of the lyophilized material can be further improved.

Examples of the hydrophobic liquid include an oil and a wax. These liquids can be used alone, however, for example, by adding a molten wax to the lyophilized material before adding an oil, and then, by adding an oil thereto after the wax is solidified, the lyophilized material can be prevented from diffusing in the oil.

Here, the wax refers to an organic material which is a liquid or a solid at room temperature, and in the case where the wax is a solid at room temperature, the solid is converted to a liquid by heating. The wax which can be used in the invention has a melting point of 31° C. or higher, preferably 36° C. or higher, more preferably 41° C. or higher, further more preferably 46° C. or higher, and is 100° C. or lower, preferably 90° C. or lower, more preferably 80° C. or lower, further more preferably 70° C. or lower, and is preferably composed of a neutral fat, a higher fatty acid, a hydrocarbon, or the like. The wax is not particularly limited, and for example, a petroleum-derived wax such as paraffin wax or microcrystalline wax, an animal-derived wax such as beeswax, wool wax, or spermaceti, or a vegetable-derived wax such as carnauba wax, rosin, candelilla wax, or Japan wax can be used. Other than these, El Christa (registered trademark, Idemitsu Kosan Co., Ltd.), Nissan Elector (registered trademark, NOF Corporation), Poem (registered trademark, Riken Vitamin Co., Ltd.), Rikemal (registered trademark, Riken Vitamin Co., Ltd.), Neo Wax (registered trademark, Yasuhara Chemical Co., Ltd.), Hi-Wax (registered trademark, Mitsui Chemicals, Inc.), Silicone Wax (trademark, Dow Corning Toray Co. Ltd.), or the like can be used.

The type of the oil is not particularly limited, and a mineral oil, a silicone oil (such as 2 CS silicone oil), a vegetable oil, or the like can be used.

Among these, as the hydrophobic liquid, at least one member selected from a silicone oil, a mineral oil, and paraffin can be contained. In particular, it is preferred to use a silicone oil.

When the hydrophobic liquid is added after the lyophilization step, it is preferred to add the hydrophobic liquid so as to fill the container in which the lyophilized material is solidified and adhered. In the case where a wax is used as the hydrophobic liquid, the wax is added in a molten state. In the case where an oil is used, it is preferred to dehydrate the oil with silica gel and/or a molecular sieve, or the like beforehand, and it is preferred to perform the addition of the oil in a glovebox or the like in which the water content is controlled to a sufficiently low level. Further, it is preferred that after the oil is added, the container is lightly centrifuged so as to remove air bubbles.

### 1.5. Method for Redissolving Lyophilized Material and Usage Thereof

In the case where the container used can be used in a nucleic acid amplification device such as a PCR device, the container in which the lyophilized material is solidified and adhered can be used for a nucleic acid amplification reaction as it is. Specifically, an appropriate amount of pure water or a buffer containing a DNA to be amplified is added so as to soak the lyophilized material, and a nucleic acid amplification reaction may be started. The amount of pure water or the buffer can be easily determined so that the final concentrations of the salts and the like are suitable for the nucleic acid amplification reaction. Incidentally, the reagent for a nucleic acid amplification reaction may be sufficiently dissolved by performing a heating treatment or a shaking treatment before the reaction.

In the case where the lyophilized material contains a reverse transcriptase, an appropriate amount of pure water or a buffer containing an RNA may be added so as to soak the lyophilized material, and in such a case, a reverse transcription reaction can be performed, and thereafter, a nucleic acid amplification reaction can be performed.

Also in the case where a hydrophobic liquid is contained, the method for dissolving the lyophilized material and the usage thereof are the same. However, in the case where a wax is used as the hydrophobic liquid and the solid wax covers the lyophilized material, the lyophilized material is heated to a temperature at which the wax is melted, and after the wax is melted, an appropriate amount of pure water or a buffer containing a nucleic acid may be added as described above. In this case, even in the case where the lyophilized material is covered with the hydrophobic liquid, the reaction occurs without being inhibited by the hydrophobic liquid.

The lyophilized material produced by the lyophilized material production method according to this embodiment can withstand long-term storage at room temperature even in a lyophilized material as it is because of including the additive in a predetermined amount, and therefore, in the case where a nucleic acid amplification reaction is performed by adding an appropriate amount of pure water or a buffer containing a DNA to be amplified after storage, the activity comparable to that before lyophilization is maintained. For example, the lyophilized material produced by the lyophilized material production method according to this embodiment realizes storage performance for nearly 2 years at a temperature of 20° C.

### 1.6. Physical Properties

The lyophilized material produced by the lyophilized material production method according to this embodiment is a porous material which is in the form of a sponge or a cake and has a lot of micropores (air holes) in which air bubbles are trapped because the method includes the preliminary freezing step and the maintaining step, and according to this, the resolubility is improved. The average opening diameter of the micropores becomes smaller when the reagent is rapidly frozen, and the resolubility and storage stability become favorable when the average opening diameter of the micropores is smaller. The average opening diameter of the pores (which is determined by, for example, measuring the diameters of the pores in the cross section a fixed number of times under a microscope, and averaging the diameters) of the lyophilized material produced by the lyophilized material production method according to this embodiment is 10 μm or less. In particular, in the case where liquid nitrogen is used as the low-temperature liquid, the average opening diameter of the pores is 5 μm or less, and thus, the lyophilized material has excellent resolubility and can be easily redissolved in about a few seconds.

In a system in which a hydrophobic liquid is added for improving storage stability to a container in which a lyophilized material of a reagent is solidified and adhered, depending on the average opening diameter of the pores of the lyophilized material, an emulsion of water and the hydrophobic liquid is formed when the lyophilized material is redissolved, and it is difficult to completely dissolve the lyophilized material. On the other hand, the average opening diameter of the pores of the lyophilized material produced by the lyophilized material production method according to this embodiment is 10 μm or less as described above, and therefore, the lyophilized material can be easily redissolved without forming an emulsion of water and the hydrophobic liquid when the lyophilized material is redissolved.

### 2. Examples

Hereinafter, the invention will be more specifically described by way of Example and Comparative Example, however, the invention is not limited only to these Examples.

### 2.1. Example 1

### 2.1.1. Preparation of Reagent to be Lyophilized

A mixed reagent solution having the following composition was prepared by adding trehalose to a reagent for a nucleic acid amplification reaction, and 1.6 μL of the thus prepared reagent was introduced into a container in the form of bowl obtained by cutting the bottom of a 2-mL test tube or a 200-μL PCR tube manufactured by Eppendorf AG.

Composition of Mixed Reagent Solution

| | |
|---|---|
| 1x SuperScript III (reverse | 0.2 μL |
| transcriptase) | |
| 5x Buffer | 2 μL |
| 10 mM dNTP | 0.25 μL |
| 20 mM forward primer | 0.8 μL |
| 20 mM reverse primer | 0.8 μL |
| 10 mM probe | 0.5 μL |
| 500 mg/mL (mass/volume) aqueous | 2 μL |
| solution of trehalose | |
| infA | 0.5 μL |
| D.W. | up to 10 μL |

Primer
Primer F:
GAC CAA TCC TGT CAC CTC TGA C
Primer R:
AGG GCA TTT TGG ACA AAG CGT CTA
Probe:
TaqMan probe:
FAM-TGC AGT CCT CGC TCA CTG GGC ACG-TAMRA

### 2.1.2. Preliminary Freezing Step

Subsequently, the PCR tube into which the reagent was introduced was immersed in liquid nitrogen and maintained for 5 minutes.

### 2.1.3. Maintaining Step

Subsequently, the PCR tube in the preliminary freezing step was placed in a deep freezer at a temperature of −80° C. and maintained for 1 hour to vaporize liquid nitrogen.

### 2.1.4. Lyophilization Step

Subsequently, the sufficiently frozen sample was quickly placed in a lyophilizer, and lyophilization was performed overnight at room temperature in a reduced pressure environment at 10 Pa, whereby a sample of Example 1 was obtained.

### 2.2. Comparative Example 1

In Comparative Example 1, a sample was prepared in the same manner as in the above Example 1 except that a PCR tube into which the reagent was introduced was placed in a deep freezer at a temperature of −80° C. and maintained for 1 hour as preliminary freezing in place of 2.1.2. Preliminary Freezing Step and 2.1.3. Maintaining Step in the above Example 1.

### 2.3. Evaluation

### 2.3.1. SEM Observation

In Example 1 and Comparative Example 1, a container obtained by cutting the bottom of a 2-mL test tube was used as the container, and the obtained lyophilized material was observed with an SEM. As the device, a low-vacuum scanning electron microscope JSM-6510LV manufactured by JEOL Ltd. was used. The observation was performed at 5 kV for the sample of Example 1, and at 1.00 kV for the sample of Comparative Example 1.

The result showed that in the sample of Example 1, the average opening diameter of micropores 10 was from 2 to 3 μm, however, in the sample of Comparative Example 1, the average opening diameter of micropores 20 exceeded 10 μm. In this manner, the result that the average opening diameters of micropores are different due to different conditions for preliminary freezing was obtained. Incidentally, the lyophilized material absorbed water during the operation of the SEM observation, and therefore, it is considered that the actual average opening diameter of micropores is smaller than the observed average opening diameter.

### 2.3.2. Redissolving Test

Next, a redissolving test was performed for a sample in which a PCR tube was used as the container, and 50 μL of a silicone oil (manufactured by Shin-Etsu Chemical Co., Ltd.) having a viscosity of 2 CS was added dropwise into the PCR tube in which the lyophilized reagent was solidified and adhered to the bottom.

The redissolving test was performed as follows. The PCR tube containing the lyophilized material was placed in a heater and the temperature was maintained at 50° C. Then, 1.6 μL of distilled water was added dropwise thereto, and the dissolution state of the lyophilized material at this time was photographed by a camera. Incidentally, as the sample, a sample immediately after lyophilization was used, and the test was performed under the conditions of n=12.

The sample of Example 1 was dissolved immediately after the liquid droplet reached the lyophilized material. Therefore, complete dissolution was confirmed by visual observation at 500 ms after the dropwise addition. On the other hand, in the case of Comparative Example 1, the lyophilized material remained in the lower portion of the container at 1 s after the dropwise addition, and moreover, the oil was caught in the inside of the liquid droplet, and a round liquid droplet was further formed in the liquid droplet. In addition, a material in the form of fine bubbles was also observed, and complete dissolution was not confirmed at 1 s after the dropwise addition. It took 20 s or more for completely dissolving the lyophilized material, and there were grains presumed to be the oil in the upper portion at 30 s after the dropwise addition.

### 2.4. Evaluation Results

From the above results, in the case of Example 1 including the preliminary freezing step using liquid nitrogen and the maintaining step, the average opening diameter of the micropores of the lyophilized material was small, and the lyophilized material was easily dissolved in a short time in the redissolving test, on the other hand, in the case of Comparative Example 1 in which as the preliminary freezing, only freezing in the deep freezer at −80° C. was performed, the average opening diameter of the micropores was larger than 10 μm, and it took time for redissolving the lyophilized material. In this manner, it was found that by including the freezing step and the maintaining step according to this embodiment, a lyophilized material which has improved resolubility and can be easily redissolved in about a few seconds can be provided.

The invention is not limited to the above-mentioned embodiments, and various modifications can be made. For example, the invention includes substantially the same configurations (for example, configurations having the same function, method, and result, or configurations having the same object and effect) as the configurations described in the embodiments. Further, the invention includes configurations in which a non-essential portion in the configurations described in the embodiments is replaced. Further, the invention includes configurations having the same operational effects or the configurations capable of achieving the same objects as those of the configurations described in the embodiments. Further, the invention includes configurations in which a known technique is added to the configurations described in the embodiments.

The entire disclosure of Japanese Patent Application No. 2015-149350, filed Jul. 29, 2015 is expressly incorporated by reference herein.

What is claimed is:

1. A lyophilized material production method, for producing a lyophilized material by lyophilizing a reagent containing an additive at a lyophilization temperature, comprising:
a freezing step in which the reagent containing an additive is cooled with a low-temperature liquid whose temperature is lower than the lyophilization temperature;
a maintaining step in which the temperature of the frozen reagent is increased to a temperature higher than the boiling point of the low-temperature liquid and the reagent is maintained at the temperature for a predetermined time; and
a lyophilization step in which the reagent having been maintained for a predetermined time after increasing the temperature of the reagent is dried at the lyophilization temperature under reduced pressure.

2. The lyophilized material production method according to claim 1, wherein the liquid is liquid nitrogen.

3. The lyophilized material production method according to claim 1, wherein the additive contains trehalose.

4. The lyophilized material production method according to claim 1, wherein the reagent contains a reverse transcriptase.

5. The lyophilized material production method according to claim 1, wherein the reagent contains at least one member selected from a polymerase, dNTP, a primer, and a probe.

6. The lyophilized material production method according to claim 1, wherein the additive is contained in an amount of 47 mass % or more and 95 mass % or less with respect to the total mass of the solids contained in the lyophilized material.

7. The lyophilized material production method according to claim 1, wherein the additive is contained in an amount of 78 mass % or more and 85 mass % or less with respect to the total mass of the solids contained in the lyophilized material.

8. The lyophilized material production method according to claim 1, wherein the method further comprising:
an addition step in which a hydrophobic liquid is added after the lyophilization step.

9. The lyophilized material production method according to claim 8, wherein the hydrophobic liquid is a silicone oil.
